(19) United States
(12) Patent Application Publication
(10) Pub. No.: US 2003/0192554 A1
(43) Pub. Date: Oct. 16, 2003

(54) Methods and apparatus for adhering musculoskeletal tissues

(76) Inventor: Bret A. Ferree, Cincinnati, OH (US)

Correspondence Address: John G. Posa, Gifford, Krass, Groh, Sprinkle,, Anderson & Citkowski, P.C., Suite 400, 280 N. Old Woodward Ave., Birmingham, MI, 48009-5394, US

(21) Appl. No.: 10/412,124

(22) Filed: Apr. 11, 2003

Related U.S. Application Data
(60) Provisional application No. 60/371,903, filed on Apr. 11, 2002.

(51) Int. Cl. A61D 1/00; A61B 17/08; A61B 19/00

(52) U.S. Cl. 128/898; 606/214

(57) ABSTRACT

A method of adhering musculoskeletal tissues such as tendons, ligaments, and cartilage involves applying one or more substances to enhance adhesion. According to one aspect, scar-forming substances are applied to the tissues that the surgeon wishes to connect. For example, tetracycline, nitrogen mustard, sterile talcum powder, erythromycin, or alternative substances may be applied to the tissues. A second aspect of the invention uses growth factors such as those found in platelet-rich plasma (PRP) to accelerate the healing. The growth factors could be sprayed onto the tissues the surgeon wishes to connect. A third aspect uses “glue” such as “fibrin glue” to bond the tissues together. Regardless of the embodiment, an inventive retractable catheter may be used to administer the agent(s) so as not to expose unintended areas.

### REFERENCE TO RELATED APPLICATION

This application claims priority from U.S. Provisional Patent Application Serial No. 60/371,903, filed Apr. 11, 2002, the entire content of which is incorporated herein by reference.

### FIELD OF THE INVENTION

This invention relates generally to spinal surgery and, in particular, for adhering musculoskeletal tissues.

### BACKGROUND OF THE INVENTION

According to current practice, musculoskeletal tissues such as tendons, ligaments, and cartilage are connected surgically with sutures and staples. Generally, the synthetic staples and sutures provide provisional fixation. Long-term connection of the tissues is dependent upon the tissues healing together, however. The natural healing process relies on the formation of fibrous scar tissue between the surgically connected tissues. In a sense, there is a race between failure of the suture and the formation of scar tissue with sufficient tensile strength to hold the tissues together. At times a patient's anatomy can make suturing or stapling of tissues difficult. For example, a surgeon may wish to connect two or more tissues inside a structure such as the intervertebral disc.

### SUMMARY OF THE INVENTION

This invention is direct to a method of adhering musculoskeletal tissues such as tendons, ligaments, and cartilage. Broadly the method involves applying one or more substances to the musculoskeletal tissues to be connected which enhances adhesion therebetween.

According to one aspect, scar-forming substances are applied to the tissues that the surgeon wishes to connect. For example, tetracycline, nitrogen mustard, sterile talcum powder, erythromycin, or alternative substances may be applied to the tissues.

A second aspect of the invention uses growth factors such as those found in platelet-rich plasma (PRP) to accelerate the healing. The growth factors could be sprayed onto the tissues the surgeon wishes to connect. A third aspect uses “glue” such as “fibrin glue” to bond the tissues together.

Regardless of the embodiment, an inventive retractable catheter may be used to administer the agent(s) so as not to expose unintended areas.

### BRIEF DESCRIPTION OF THE DRAWINGS

FIGS. 1A and 1B are drawings which show a retractable catheter according to the invention;

FIG. 2A shows a coronal cross-section of a retractable catheter and syringe; and

FIG. 2B shows a coronal cross-section of a retracted catheter and syringe shown in FIG. 2A.

### DETAILED DESCRIPTION OF THE INVENTION

This invention addresses the above problems associated with sutures or staples. According to one aspect, scar-forming substances are applied to the tissues that the surgeon wishes to connect. For example, tetracycline, nitrogen mustard, sterile talcum powder, erythromycin, or alternative substances may be applied to the tissues. The scarring affects of tetracycline and nitrogen mustard are well known to surgeons who perform pleurodesis (for the lung). This method could be used to “glue” tissues of the intervertebral disc together using, for example, adhesives such as Dermabond and those used in Dentistry.

A second aspect of the invention uses growth factors such as those found in platelet-rich plasma (PRP) to accelerate the healing. The growth factors could be sprayed onto the tissues the surgeon wishes to connect. A third aspect uses “glue” such as “fibrin glue” to bond the tissues together.

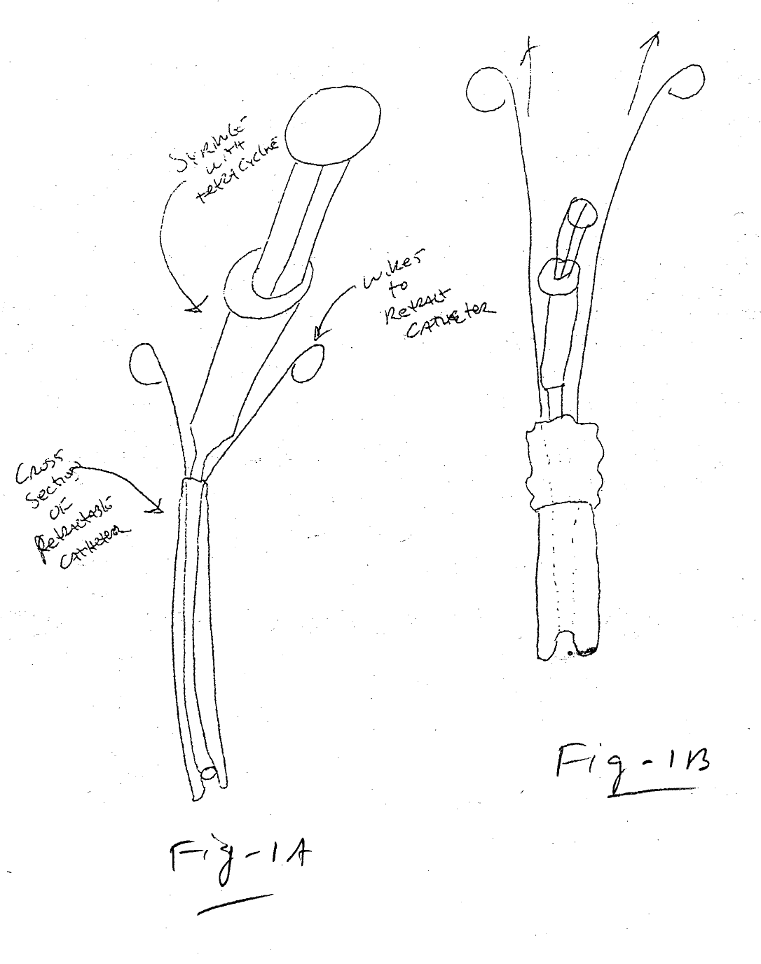

Regardless of the embodiment, an inventive retractable catheter may be used to administer the agent(s) so as not to expose unintended areas. As shown in FIG. 1A, a syringe is coupled to the catheter in an extended state to deliver the substance(s), which is retracted after delivery (FIG. 1B).

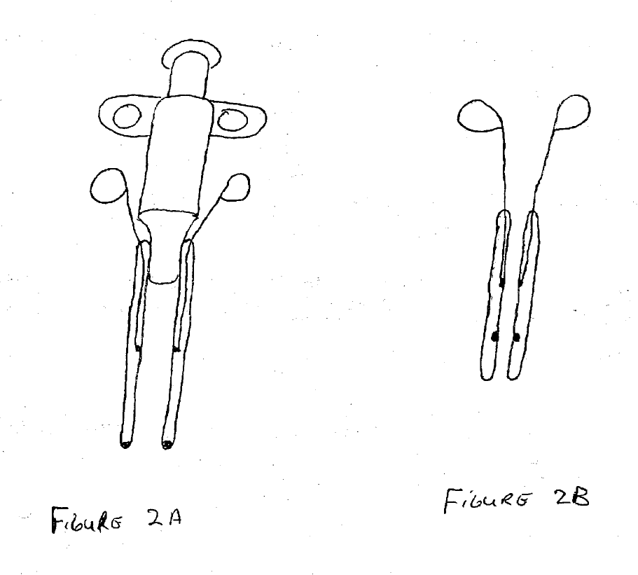

FIG. 2A is a coronal cross section of the retractable catheter and a syringe according to the invention. The catheter can be retracted by pulling on the inner walls of the catheter. For example, wires can be attached to the inner walls of the catheter to pull the catheter into itself.

FIG. 2B is a coronal cross section of a retracted catheter of the embodiment drawn in FIG. 2A. Please notice the dark circle on the end of the extended catheter of FIG. 2A is inside the retracted catheter of 2B.

I claim:

1. A method of adhering musculoskeletal tissues such as tendons, ligaments, and cartilage, comprising the step of:
applying a substance to the musculoskeletal tissues to be connected which enhances adhesion therebetween.

2. The method of claim 1, wherein the substance is a scar-forming substance.

3. The method of claim 2, wherein the substance is tetracycline.

4. The method of claim 2, wherein the substance is erythromycim.

5. The method of claim 2, wherein the substance is nitrogen mustard.

6. The method of claim 1, wherein the substance is a growth factor.

7. The method of claim 1, wherein the growth factor is found in platelet-rich plasma (PRP).

8. The method of claim 1, wherein the substance is a fibrin glue.

9. The method of claim 1, further including the step of using a retractable catheter to administer the substance so as not to expose unintended areas.
